(19) United States
(12) Patent Application Publication
(10) Pub. No.: US 2009/0062869 A1
(43) Pub. Date: Mar. 5, 2009

(54) MINIMALLY INVASIVE BONE FIXATION CLAMP FOR NAVIGATED SURGERIES

(75) Inventors: Frederic Pierre Claverie, Seyssins (FR); Christopher Plaskos, New York, NY (US)

Correspondence Address: Leason Ellis LLP, 81 Main Street, Suite 100, White Plains, NY, 10601, US

(73) Assignee: Perception Raisonnement Action en Medecine, La Tronche (FR)

(21) Appl. No.: 12/199,955

(22) Filed: Aug. 28, 2008

Related U.S. Application Data
(60) Provisional application No. 60/968,370, filed on Aug. 28, 2007.

(51) Int. Cl. A61B 17/04; A61B 17/08

(52) U.S. Cl. 606/324; 606/151

(57) ABSTRACT

In one embodiment, a device for fixing a first object to a second object during a navigated surgery includes a body; a pair of articulating arms that are pivotally connected to the body; and a pair of deformable, elastic components that are detachably coupled to an inner face of the articulating arms so as to permit removal and replacement of the components relative to the articulating arms. An exposed surface of the elastic components is intended to contact the second object to permit the device to be securely attached to the second object.

### CROSS-REFERENCE TO RELATED APPLICATION

The present application claims the benefit of U.S. patent application Ser. No. 60/968,370, filed Aug. 28, 2007, which is hereby incorporated by reference in its entirety.

### TECHNICAL FIELD

The present invention relates to a means for fixing a medical device onto a patient's bone during a navigated surgery and in particular, relates to a means of improving medical clamps that are used during navigated surgery

### BACKGROUND

Computer Assisted Orthopedic Surgery (CAOS), or navigation, requires the surgeon to fasten onto the patient's bones a medical device called a “reference” in order to allow the navigation system to localize the patient's bones in space. These references can be retro-reflective markers, active light emitting diodes, ultrasound emitters, radiofrequency, electro-magnetic, or magnetic transponders etc., depending on the technology used by the system. References can also be mechanical localizers, arms, bone immobilizers, or bone motion sensors, etc. . . . as are commonly used in robotic surgery. This fixation has to be as strong as possible for if the reference moves relative to the bone during the surgery, the system looses track of the patient's bone (i.e., looses registration).

Current means for fastening theses references include: clamps, screws and threaded pins (broaches), and nails. Each of these devices is discussed below.

(A) Clamps: Clamps are usually used for fastening references onto small bones, such as, lumbar or cervical vertebrae, though they have been also applied to larger bones, such as, the distal and proximal femur, humerus etc. Clamps rely on spikes placed on opposite sides of the bone. Clamps have the following disadvantages: (1) their geometry doesn't always conform to each patient's anatomy and as a result certain spikes can be in contact with the bone while others can still be in mid air; (2) clamps do not balance the pressure applied on each spike because the jaws are not articulated to do so; and (3) clamps do not limit the strength that the surgeon is able to apply to the clamp since they usually rely on screws to fasten the clamp and consequently, the surgeon has very little indication of the strength that is being applied to the bone. These disadvantages can lead to situations where the spikes penetrate the bone so deeply and so unevenly that it results in a partial or complete fracture of the bone.

FIG. 1 shows a conventional clamp design.

(B) Threaded pins and screws: This category includes bi-threaded pins and mono-threaded pins. They are commonly used for fastening references onto larger bones such as femur, pelvis, tibia, shoulder etc. Threaded pins and screws have the following disadvantages: First, their effectiveness depends largely on the bone quality. In cancellous bone, the fixation strength is weakened. This problem was partially solved by creating specific thread profiles for cancellous bones but the damage to the bone remains. To the contrary, in hard cortical bone the compression they create around the insertion site can cause fractures. This problem was partially solved by drilling a pre-hole before inserting the broach but the damage to the bone remains. Another disadvantage is that a failing fixation site means that the surgeon has to drill more holes into the bone. This process can not be repeated endlessly so the surgeon must get it right at the first time. One more disadvantage is that the surgeon has to be cautious not to drill through the bone into critical soft tissues. The only feedback the surgeon gets is the bone resistance felt while screwing or drilling which is likely to be misinterpreted or missed altogether.

C. Nails: Nails can be triangular in cross-section or they can have any number of edges running down the length of the nail to prevent rotations. They are directly hammered into the bone. Disadvantages of nails include: (1) they are very likely to create a stress concentration and fracture and nothing prevents them from skidding out of the bone. This problem was partially solved by drilling a pre-hole but the same disadvantages as the threaded pins remain; (2) they cannot be used on small bones for they would break them right away; and (3) there is a risk of punching them all way through the bone into critical soft tissues behind the bones. The scope of use of nails is therefore limited, they are risky and their use requires considerable skill from the surgeon.

As a conclusion, all these systems damage the bone and are prone to creating fractures if the surgeon applies too much force. They are not forgiving for mistakes from the surgeons and can create unnecessary damage to the patient, leading to potentially more post operatives problems.

### SUMMARY

The present invention relates to an interface for bone clamps in the surgical field. The interface provides a strong and reliable fixation to bones while minimizing bone damage and risk of fracture. It achieves this result by making use of programmed-deformation parts, that could be made out specific materials, such as plastic, and they could be combined with articulating joints or jaws, wherein the operating function is to limit and balance the force that the surgeon applies to the bone while retaining as much friction as possible.

The objects and advantages of the interface of the present invention are: (1) it allows the surgeon to fix rigidly a medical device onto a bone; (2) it improves the rigidity of fixation over the prior art using principles of material deformation and contact friction between the bone and device; (3) it minimizes the damage done to the bone, and more importantly it minimizes the risk of fracturing the bone by: (a) balancing the pressure over a greater surface area, among several contact areas and spikes; (b) limiting the peak force applied directly to the bone by the surgeon by spreading it evenly through a surface area that increases with the applied force; (c) limiting the spike penetration into the bone; and (4) it can be applied to any kind of surgical bone clamp dedicated to any kind of bone.

The interface of the present invention also provides a shock absorbing feature such that if the clamp is bumped, the force is taken up by one or more of the deformable elastic components, and not transferred to the bone surface, potentially irreversibly damaging the bone surface and compromising the fixation rigidity.

The device of the present invention is also configured such that it is simple to assemble, it reduces the risks of patient infection, and is flexible in the sense that it allows the manufacturer to reduce the number of components required to keep in stock

In one embodiment, a device for fixing a first object to a second object during a navigated surgery includes a body; a pair of articulating arms that are pivotally connected to the body; and a pair of deformable, elastic components that are detachably coupled to an inner face of the articulating arms so as to permit removal and replacement of the components relative to the articulating arms. An exposed surface of the elastic components is intended to contact the second object to permit the device to be securely attached to the second object.

In another embodiment, a clamp for fixing a first object to a second object during a navigated surgery includes a body; a pair of first pivotal members that are pivotally attached to the body; and a pair of articulating arms that are pivotally connected to the first pivotal members. Each articulating arm has a pair of support structures. The clamp also includes a pair of deformable, elastic components that are detachably coupled to an inner face of the articulating arms so as to permit removal and replacement of the components relative to the articulating arms. Each elastic component engages the support structures resulting in the elastic component being carried by the articulating arm.

Further objects and advantages of the invention will become apparent from a consideration of the drawings and ensuing description.

### BRIEF DESCRIPTION OF THE DRAWING FIGURES

FIG. 1 is a perspective view of a conventional clamp device;

FIG. 2A is a perspective view of a bone clamp for the vertebrae according to one embodiment of the present invention;

FIG. 2B is a perspective view of the clamp secured onto a spine vertebra;

FIG. 3 is an exploded view of the articulated jaw clamp assembly;

FIG. 4 is a cross-sectional view of the articulated jaw feet of the clamp;

FIG. 5A is an enlarged sectional view of an exemplary bone/clamp interface with low clamping force;

FIG. 5B is an enlarged sectional view of an exemplary bone/clamp interface with high clamping force;

FIGS. 6A-C are enlarged sectional views of an exemplary bone/clamp interface with a suction force;

FIG. 7 is a perspective side view of a bone clamp for a long bone, such as a femur or humerus, according to one embodiment of the present invention;

FIG. 8 is an exploded perspective view of the articulated arm and jaw clamp assembly of the clamp of FIG. 8; and

FIG. 9 is a cross-sectional view showing details of the contact feet and pattern of the clamp.

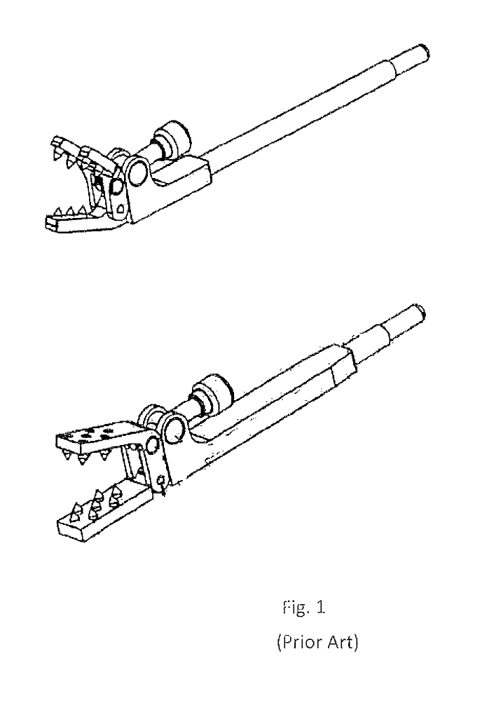

### DETAILED DESCRIPTION OF EXEMPLARY EMBODIMENTS

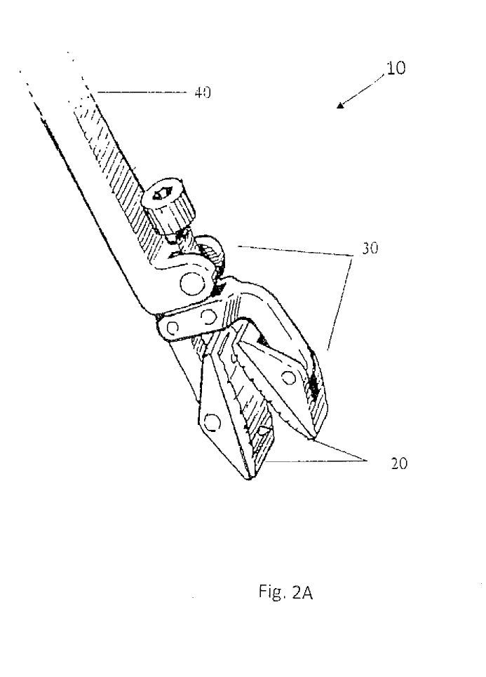

Referring now to FIG. 2A, a bone clamp 10 of the present invention is illustrated. The clamp 10 consists primarily of three components: 1) one or more deformable elastic interface components 20 for contacting the bone surface to which the device will be fixed to; 2) an articulating component 30 for orienting and/or clamping the deformable interface component to the bone surface; and 3) a body portion 40 that is intended to fix a reference marker or tracker to.

The reference tracker (not shown), can be of any type known in art of computer- or robot-assisted surgery, and can be attached to the clamp using additional articulating and fixing elements (for example, if an optical tracker is used, it can be oriented and locked to face the camera of the computer assisted surgery system using an additional articulated connection means). Alternatively, the reference tracker can be integral to the clamp body itself. For example, the clamp could be made entirely out of plastic and a magnetic tracker could be directly integrated or molded into the plastic body. The entire assembly can be manufactured using low-cost methods and intended for single-use (disposable).

The tracking device could also be mechanical in nature (for instance, if a medical robot is used instead of or in combination with a navigation system), such as a mechanical arm that is equipped with encoders or sensors and that is connected to the robot, allowing to robot to know where the bone is located relative to the robot's coordinate system. The clamp may also be used in combination with an immobilization system, to minimize or prevent the patient from moving as much as possible during the surgery. The body portion 40 of the clamp could be connected to a rigid or flexible arm or holder that is attached to the table or robot base to stabilize the motion of the bone, with a tracking device to monitor any small bone motions.

As can be seen in FIG. 2B, this particular embodiment (clamp 10) is intended and has been designed for concave bone surfaces, such as, but not exclusively, the spinous process of a vertebra. In other words, the feet of the deformable interface elements 20 are designed with a convex surface shape (i.e. bulging outward) to better mate with the generally concaved bony anatomy that is expected to be encountered at the fixation site during use.

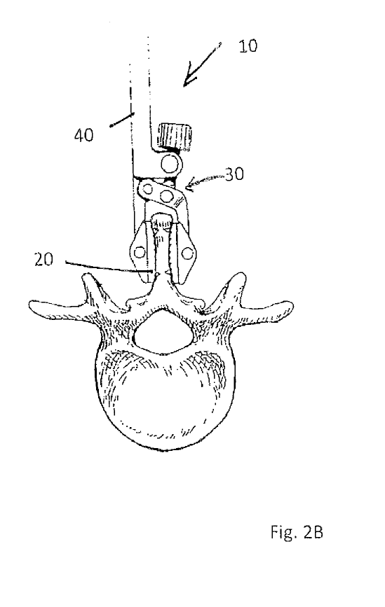

Turning now to the exploded perspective view of FIG. 3, it can be seen that the clamping device 10 is composed of the body 40 that is coupled with a clamping mechanism. A clamping screw 42 and mechanism applies a force onto a mobile arm 44. The mobile arm 44 is connected to the body 40 by means of an axle or pin 46 such that the mobile arm 44 pivots about an axis extending through pin 46. It is presently preferred that this connection is made by means of pin 46, but the movement of the mobile clamping element (arm 44) relative to the body 40 can be a translation motion instead a rotation, or a combination of the two. In this case, the connection between the mobile arm 44 and the body 40 would be different in form and function.

The mobile arm 44 is connected to an articulated arm 14a by means of an axle or pin 13a that defines an axis extending therethrough. Symmetrically, another articulated arm 14b is connected to the body 40 by means of an axle or pin 13b that defines an axis extending therethrough. It is presently preferred that this connection be made with a pin or the like having a diameter of approximately 3 mm and that the pin is made of stainless steel. This leaves one degree of freedom between the articulated arm 14a and the mobile arm 44. However, this connection could also be done with other type of joints, such as, a ball joint whose size could range from 1 to 10 mm and the material used to make the ball joint can be plastic (such as PEEK, PEI or ULTEM) or another metallic alloy (such as Titanium TA6V). Deformable ‘flexure’ joints can also be used, in which the material bends under the applied load. The overall geometry or the cross-section of the material used to make the flexure joint can be optimized so that the bending is carried out primarily in one or two directions, depending on the expected location of use and size constraints. The device could also be manufactured partially or completely out of radio-transparent or non-magnetic materials for use with intra-operative imaging modalities, such as fluoroscopic C-arms, CT, or MRIs.

Two pairs of metallic spikes 16a, 16b, and 16c, 16d, are welded on each articulated arm 14a and 14b. These spikes can be laser welded to the respective articulated arm 16a, 16b, 16c or 16d, or they could be machined out of the material directly, or assembled by any other means (e.g., press mounting). Each spike 16a, 16b, 16c, 16d has a cone 19 (e.g., 60 degree cone) and a tip. In one example, the cone 19 has a height of 1 to 2 mm and the base cone diameter is 2 mm. The shape, number and layout of the spikes can be different depending on the embodiment. Each articulated arm 14a, 14b can have from 1 to 6 spikes, for example, or even more if the intended use was on a large bone, such as a femur. Of course, the spikes can also be omitted completely, as they are only intended to supplement the fixation provided by the deformable elements, and in particular, to help prevent the clamp 10 from sliding on the bone surface.

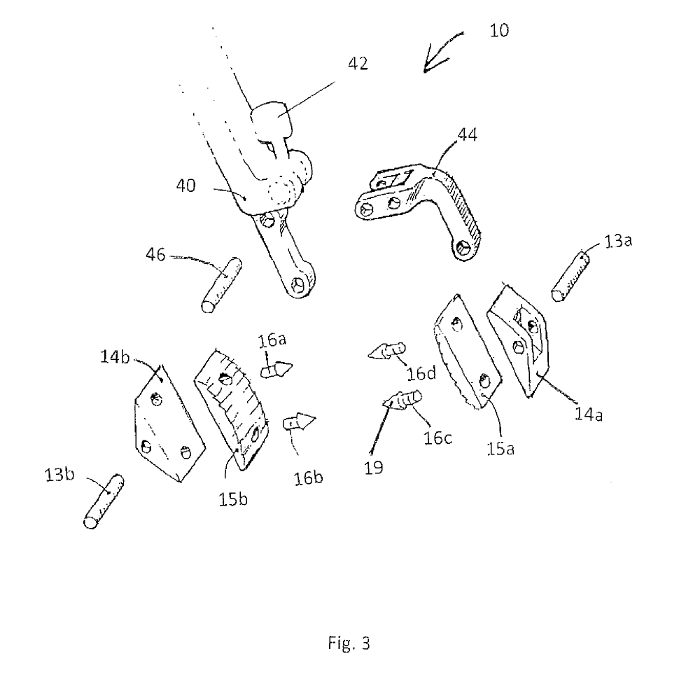

To provide a deformable elastic interface component plastic pads 15a and 15b are clipped onto each articulated arm 14a and 14b, respectively. FIG. 4 shows a detailed view of one possible clipping principle in which the pad 15 is clipped and held onto the articulating arm 14 using the spikes 16. Each plastic pad 15 features two holes to allow the spikes 16 to go through it, extending roughly 2 mm beyond the pad surface. However, the pads 15 can also be clipped directly onto the articulated arms 14. In particular, a clip mechanism (not shown) that does not protrude through the outer pad surface can be provided, such as dedicated low-profile pegs in the center area or an inward beveled lip or other specific edge shape that surrounds the perimeter of the arm 14 and mates with the pad 15. Providing the deformable elements in the form of pads 15 that can be clipped onto the clamp 10 allows the pads 15 to be easily replaced when they are damaged or worn.

The clamp 10 can be provided in metal or another rigid material so that it can be reused for a long period of time, saving costs as the hospital only needs to replace a small portion of the clamp 10 and not the entire mechanism, which can be more costly to produce. New single-use pads may be provided and packaged sterile and ready to use, and they can be included as part of a disposable navigation kit that contains other elements required to perform the navigated surgery, such as disposable retro-reflective or magnetic markers for the trackers, software start cards and/or CD-ROMs for starting the application software and saving a navigation report on, as well as instructions for use. Thus, the clamp 10 can be provided in a hybrid format with multiple-use (more costly) and single-use (low-cost) components that are replaced before every procedure. Since the pads 15 can be optimized for the particular anatomical region, the same reusable clamp design can be used for different surgical applications or in various anatomical regions simply by clipping on the appropriate pad 15. Replacing the part (pad 15) that is in direct contact with the patient with a new sterile one can also reduce the risk of infection and cross-contamination.

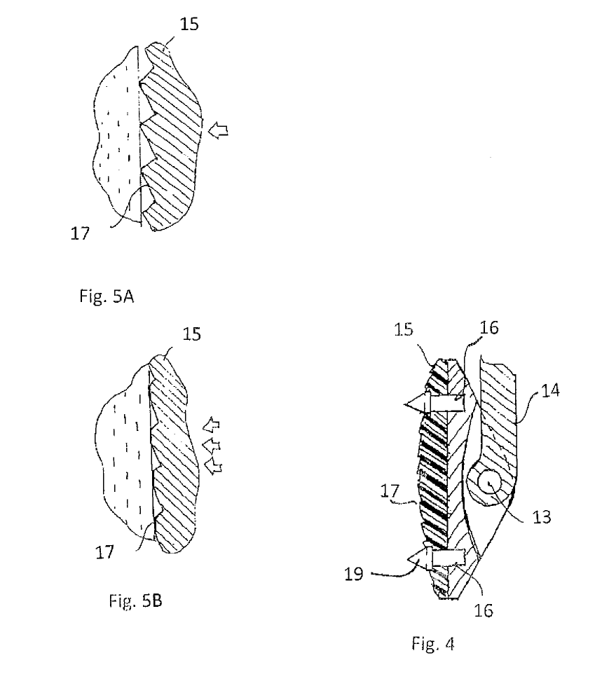

In an embodiment of the present invention shown in FIGS. 5A and 5B, a special surface 17 is embossed or incorporated on the pad 15. This special surface 17 undergoes a predicted, engineered, or programmed deformation pattern when the clamping force is applied. As can be seen in FIGS. 5A and 5B, the surface 17 can have a pattern of ridges that deform as the load applied to the articulating arms (not shown) is increased (see FIG. 5B). As the applied load is increased, the contact surface area may also increase, better distributing the loads and increasing the frictional area, helping to preventing the pad from sliding on the bone surface, say for example if it were accidentally bumped. The directional elasticity of the material can also be configured such that when sufficient clamping compression is applied, the flexible pattern or teeth become stiffer in the direction tangent to the surface (to increase lateral rigidity). A structure with space incorporated between the flexible teeth (surface 17) can be provided, this space completely filling up by the deformed teeth at a predefined pressure, reducing the tendency of the teeth to flex laterally when lateral forces are applied. The deformation pattern can also be programmed to provide a visual indication to the surgeon when he/she has applied sufficient load to assure proper fixation (for example, a cavity in the material collapses under a predefined load). The pad 15 can be made out of a plastic that has a carefully chosen hardness. In one embodiment, the pad 15 can have a hardness ranging from 30 to 100 Sha; however, it will be understood that the dimensions and geometry of the pads 15 have to be adapted to the material. Acceptable materials include, but are not limited to, ABS, PEhD, Silicone, Fluorocarbon, Chlorobutyle, EDPM, PP, EVC, LSR, RTV2, EVA, EVOH.

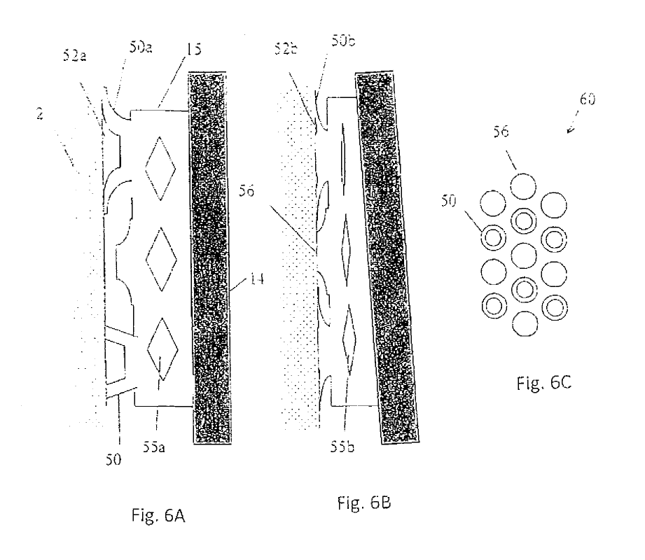

Referring now to FIGS. 6A-C, another embodiment of the present invention is illustrated. Similar to an octopus' tentacles, small or micro-sized suction-cups 50 are incorporated into the deformable interface (pad) 15 in order to better grip and help prevent slipping on the bone surface 2. The tiny cups 50 can come in various forms (constructions and configurations) 50 50a, and can be combined with other geometric forms, such as dots 56 to form a pattern 60 as shown in FIG. 6C. When the micro-cups 50 come into contact with the bone surface 2 and are deformed 50b (FIG. 6B) under the applied clamping load, the volume 52a (interior space) in the cup 50a is reduced as indicated at 52b in FIG. 6B, air and bodily fluid are forced outside the cup walls, and a partial vacuum is created. Using very small cups 50, 50a can help prevent any larger irregularities in the bone surface 2 from letting air inside the cup 50, 50a and diminishing the suction effect. Fluids, such as, water, saline, blood, etc., can also help to provide an improved seal. The dots 56 can provide additional stiffness and rigidity to the fixation, while helping to distribute the contact load, combining the advantages of both features. Multiple layers of material with different structures and/or properties can also be incorporated to benefit from different structural or material characteristics. For example, cellular or porous materials can be used such that when the material is compressed, pours deform, with the deformation being proportional to the load applied. FIG. 6A shows a porous space 55a in a relaxed state when no load is applied, while FIG. 6B shows the pore deformed (compressed) as indicated at 55b.

In another embodiment, the deformable interface (pad) 15 is integral to the articulating arm (i.e., not detachable). The deformable interface 20 can be manufactured out of the same material as the articulating arm, preferably by injection molding, and can have an optimized geometrical structure such as teeth, suction cups, etc., directly incorporated to allow for surface deformations at the level of the bone interface. The articulating arms can be designed to flex, and a screw can be inserted incorporated such that when the screw is turned the arms are bent. The body portion can also be integrated by injection molding. The deformable surface can also be manufactured out of a different material and incorporated into the arms using a bi-injection process or a secondary manufacturing process. Such processes are well known in the manufacturing industry (for example, ergonomic tooth brush handles with integrated grips). Thus the entire clamp can be manufactured to be low cost and in one piece, and the entire construct can be provided as a single use device.

Smart materials or advanced polymers can also be used in the deformable elastic interface components 20, where the same material can have both elastic and rigid or semi-rigid states (for instance, a material having an adaptive composite structure, such as shape memory polymer (SMP) or foam). The transition from rigid to elastic could be triggered by applying energy such as thermal energy (heat) to the material before applying the clamp, the amount of heat being less than that known to cause bone necrosis. In the elastic state the polymer easily deforms to accommodate the surface of the bone when the clamping load is applied, so that the material is almost entirely in contact with the bone. The material then cools down in a relatively short amount of time (approximately one minute or preferably less), becoming stiffer while still conforming to the bone shape, better distributing contact pressures on irregular bone surfaces. The articulating arms and the applied clamping force can take up any residual play or relieve any residual stress caused by shrinkage or expansion of the polymer during hardening.

The deformable elastic components, such as cups 50, of the system can also function as a ‘shock-absorber’ if the reference marker is accidentally bumped during the surgery. In the case of a completely rigid system, all of the force from the bumping action is transferred to the bone fixation site, possibly damaging or crushing the bone surface in the contact area. As the shape of the bone is now altered, a rigid system would not be able to adapt, and rigidity (and thus registration and tracking) would be lost. In a flexible system, the transmitted force is absorbed up by the deformable elastic components, or the flexure joints in the articulation system, and returned in the form of kinetic energy. The reference marker therefore springs back to its original position, maintaining fixation and rigidity and registration.

This shock-absorbing function could also be carried out by, or supplemented with, an additional flexible member positioned anywhere in the mechanical chain connecting the bone and the reference marker, that is dedicated for this purpose. The shock-absorbing system could include either a passive magnet, flexible member, or a spring element that connects the clamp body portion 40 with the reference marker, holding them together with a spring force. With an optical marker system, where the reference markers are typically larger and more cumbersome, this spring element is preferably used in combination with, or incorporated directly into a reproducible connection system, such as a specialty designed reproducible quick-connect system. A reproducible connection system with accurately manufactured reference surfaces allows two parts to be coupled and decoupled (mated) reproducibly, so that the two objects always maintain the same unique relative position when coupled. This allows the reference markers to be removed with the CAOS system is not tracking the bone, giving the surgeon more space to operate on the patient. The spring force can be positioned between the two connection reference surfaces to keep the two reference surfaces in contact with each other, but to allow one surface to temporarily lift off the other when the reference marker is bumped with a force that is large enough to overcome the spring force.

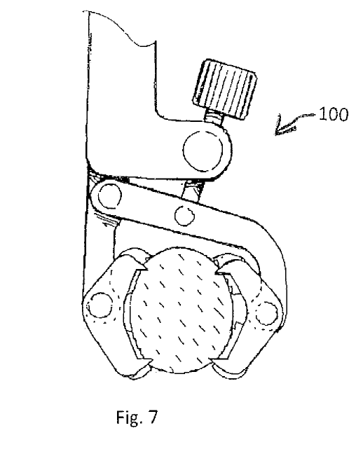

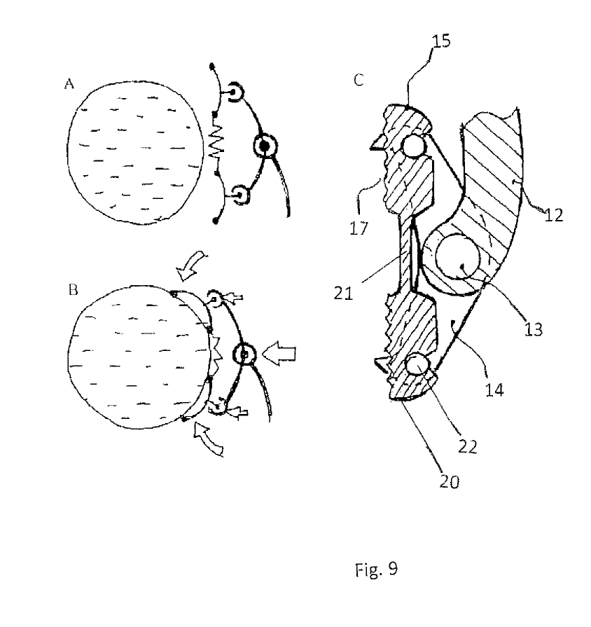

Referring now to FIGS. 7-9, several other embodiments of the bone clamp are illustrated and are generally indicated at 100. This clamp 100 can be applied to larger bones, such as, the distal or proximal femur (e.g., on the greater trochanter) or humerus, radius, etc. The invention can be applied to several different areas of bones, and as such can be used in several different types of navigated surgeries. These include joint replacement or resurfacing procedures such as those performed in the knee (eg by clamping on one femoral condyle or on the distal aspect of the femur), in the hip (eg. clamping on the neck of the proximal femur), in the shoulder, etc. . . . As can be seen in the exploded view, the pads 15 are supported by two structures 22 (e.g., pins) each, so that the load transfer is directed to different portions of the pad 15. This is better illustrated in FIG. 9. The pad 17 is made from two flexible feet portions 20 and an intermediate articulating portion 21. Each foot 20 is clipped onto and supported by an axle or pin 22, and is free to pivot about the axis defined by pin 22. When the articulated arm 14 comes into contact with the bone, it rotates so that all spikes are in contact with the bone. When a load is applied to the articulated arm 14 via the mobile arm 13, this load transferred to pads via the axes 22, causing each flexible portion 20 of the pad 15 to deform and completely grip the bone surface, the intermediate portion 21 also deforming to accommodate the rotation of the feet 20. The intermediate portion 21 can also act as a spring element which applies additional directional force on the feet 20, helping to increase function and maintain contact with the bone surface. The clamping force is transferred to the bone primarily or even totally via the deformed feet 20, which prevents the spikes from entering too far into the bone and fracturing it. This is one example of how a deformable flexible elastic interface element 17 can be ‘programmed’ to deform so that it optimally matches the bone shape and distributes the load. Programmed deformations can be used at both the micro-scale (i.e., micro deformations of textured surface pads, etc.) and the macro-scale (i.e., larger shape deformations of pads, joints, flexible members, flexures, etc.) This principle can be also extended to 3D, by orienting deformations, axes, and articulations out of the plane of the page.

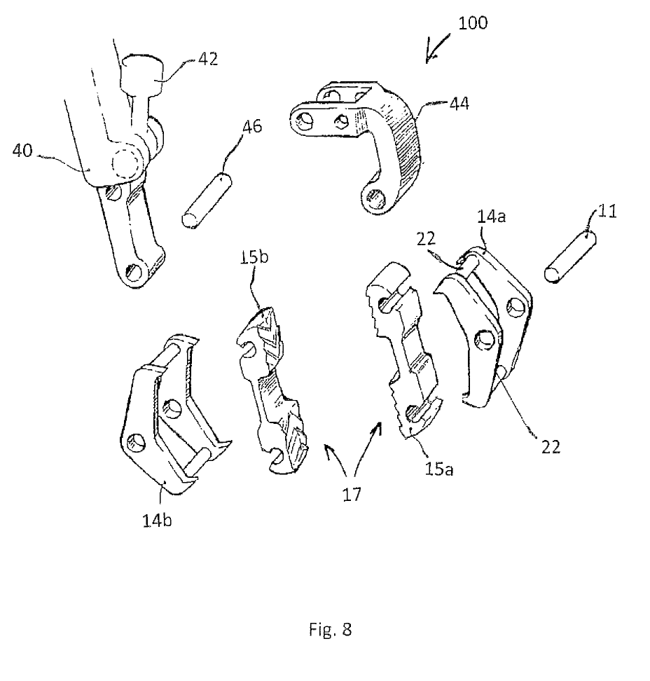

FIG. 8 shows a design where the pads 15a, 15b are snap-fit onto the pins 22 due to the pads 15a, 15b having a recessed channel formed therein for receiving the pin 22 therein so as to couple the pads 15a, 15b onto the arm using a mechanical fit (e.g., snap-fit or frictional fit) that still permits the necessary flexing action of the pads 15a, 15b. FIGS. 9A and 9B show other embodiments, wherein the two feet are attached with a bellows structure or the like that permits the desired degree of movements as with the hinge 21 of FIG. 9C.

Several variations of the invention can be envisioned. Some examples of these are illustrated in the Appendix A that is attached hereto. For instance, the arm and the pad could be made in one piece, and clipped onto the clamping arms. Empty spaces can be provided in the material to allow for deformations. Jaws can be spring loaded. Pressure can be applied to multiple points of a pad to better distribute contact stresses using linkages or deformable mechanisms. Various forms of friction patterns can be imagined, including ones inspired from shoes and tires.

The principle could be applied to any bone in the body (bones of the skull, hand, wrist, ankle, feet, pelvis, iliac crest, etc.). The interface components 20 for contacting the bone surface can be designed to be low profile with curved articulating arms that are inserted through a small incision to optimize the surgical approach for the particular surgery and to minimize the amount of soft tissue dissection required.

The operation of the clamps of the present invention will now be described. The surgeon places the clamp 10 on the vertebrae as represented on FIG. 2B and holds it roughly in the desired position. The surgeon then fastens it onto the bone using the clamping mechanism (e.g., an H3.5 orthopedic screwdriver). Because the articulated arms 14a, 14b are linked to the body 40 and the mobile arm 44 by their middle point, they orient themselves in the optimum position as soon as the spikes 16 touch the bone.

As the surgeon increases the clamping force, the spikes 16 penetrate deeper and deeper into the bone until the plastic pads 15 reach contact with the bone. From this point onward, the spikes 16 won't go much deeper into the bone. As the surgeon continues to fasten the clamp, the plastic pad 15 is more and more compressed. The pattern on the pad surface flattens and the contact surface with the bone increases as shown in FIGS. 5A and 5B. This increasing surface counter balances the increasing clamping force and the pressure applied to the bone remains roughly the same. As shown in FIGS. 5A and 5B, the pattern on the pad surface is specifically designed to flatten in a linear way as the pressure increases. This is the reason why it is called programmed deformation pattern. More importantly, the spikes 16 do not penetrate the bone too deeply and the pressure is evenly balanced on a large surface due to the plastic pad 15 and the arm's articulations.

It will be appreciated that the clamping interface of the present invention provides a secure way of fastening a medical device onto a bone. It reduces dramatically the risk of fracture by spreading the clamping force throughout a surface rather than concentrating it onto a few points. It is minimally invasive in the sense that the damage done to the bone is almost reduced to nothing: Indeed, instead of having to drill holes in the bones this system will only leave a couple of small dots on the bone surface which is a major improvement over prior art. Finally its building principle makes it fool proof for surgeons who might apply too much force to the clamp.

While the above description contains a number of specificities, these should not be construed as limitations on the scope of the invention, but rather as an exemplification of one preferred embodiment thereof. Many other variations are possible. This coupling principle can be successfully and easily applied to many different bone clamps dedicated to many different bones such as Femoral neck, Shoulder coracoids, Lombard of cervical vertebrae, etc. Any clamping mechanism can be used with this system. Its use is not limited to navigated surgery but to any surgery where the surgeon needs to fasten a device (for example, any type of sensor, or actuator including a miniature bone mounted robot) rigidly to the patient's bones.

Accordingly, the scope of the invention should be determined not by the embodiments(s) illustrated, but by the appended claims and their legal equivalents.

What is claimed is:

1. A device for fixing a first object to a second object during a navigated surgery comprising:
a body;
a pair of articulating arms that are pivotally connected with respect to one another and are connected to the body; and
a pair of deformable, elastic components that are detachably coupled to an inner face of the articulating arms so as to permit removal and replacement of the components relative to the articulating arms, wherein an exposed surface of the elastic components is intended to contact the second object to permit the device to be securely attached to the second object.

2. The device of claim 1, wherein the articulating arm is pivotally connected to a mobile arm that is itself pivotally connected to the body

3. The device of claim 2, wherein the mobile arm is pivotally connected to the body via a first pin about which the mobile arm pivots and the articulating arm is pivotally connected to the mobile arm via a second pin about which the articulating arm pivots.

4. The device of claim 1, wherein each deformable, elastic component has an exposed, inner surface that has a convex shape.

5. The device of claim 1, further including a plurality of rigid coupling protrusions extending outwardly from the inner face of the articulating arms for reception through complementary through holes in the elastic components so as to attach the component to the articulating arm.

6. The device of claim 5, wherein the protrusion has a base portion and a conically shaped distal end, wherein when the elastic component is attached to the articulating arm, the elastic component is disposed between the conically shaped distal end and the inner surface of the articulating arm.

7. The device of claim 1, wherein the deformable elastic component is carried by the articulating arm and can be removed therefrom without requiring the articulating arm to be detached from the body.

8. The device of claim 1, wherein the device comprises a clamp.

9. The device of claim 1, wherein the first object is a medical device and the second object is a bone.

10. The device of claim 1, wherein the exposed surface of the elastic component is constructed so that is undergoes a predicted deformation pattern when a force is applied to the elastic component.

11. The device of claim 10, wherein the exposed surface includes a plurality of deformable teeth.

12. The device of claim 10, wherein the exposed surface includes a plurality of suction cup structures, each suction cup structure having a hollow interior space that permits deformation of a surrounding wall of the structure when a force is applied to the elastic component.

13. The device of claim 12, wherein the exposed surface further includes a plurality of solids protuberances extending outwardly from the inner surface and interspersed with the suction cup structures.

14. The device of claim 10, wherein the elastic component is formed of a cellular material and includes a plurality of porous spaces formed throughout the elastic component below the exposed surface, wherein when the force is applied to the elastic component) a volume of the porous spaces is reduced due to compression of the elastic component.

15. The device of claim 14, wherein the porous spaces are air pockets formed throughout the elastic component.

16. The device of claim 1, wherein the elastic components comprise pads that are clipped onto the articulating arms.

17. The device of claim 10, wherein the predicted deformation pattern provides a visual indication to the user when the user has applied a sufficient load to assure proper fixation of the device to the second object.

18. The device of claim 17, wherein the visual indication comprises the collapse of a cavity formed in the elastic material of the component.

19. A clamp for fixing a first object to a second object during a navigated surgery comprising:
a body;
a pair of first pivotal members that are pivotally attached to the body;
a pair of articulating arms that are pivotally connected to the first pivotal members, each articulating arm having a pair of support structures; and
a pair of deformable, elastic components that are detachably coupled to an inner face of the articulating arms so as to permit removal and replacement of the components relative to the articulating arms, each elastic component engaging the support structures resulting in the elastic component being carried by the articulating arm.

20. The clamp of claim 19, wherein the wherein the support structures comprise a pair of pins and the elastic component has a pair of recessed channels that permit the elastic component to be clipped onto the articulating arm by inserting the pins in the channels.

21. The clamp of claim 19, wherein each elastic component includes a pair of flexible feet portions attached to one another by an intermediate articulating portion in the form of a living hinge.

22. The clamp of claim 21, wherein each of the feet portions pivots about one support structure.

23. The clamp of claim 19, wherein an exposed surface of the elastic component is constructed so that is undergoes a predicted deformation pattern when a force is applied to the elastic component.

24. The clamp of claim 23, wherein the deformation pattern optimally matches the shape of the second object to which the clamped device is attached by means of the articulating arms and distributes the applied load over the elastic component.

25. The clamp of claim 19, wherein the articulating arm is pivotal connected to the first pivotal member by a first main pin that is disposed between the pair of pins that define the support structure

26. The clamp of claim 19, wherein the first object comprises a medical device and the second object comprises a bone.

27. A device for fixing a first object to a second object during a navigated surgery comprising:
a body;
a pair of articulating arms that are pivotally connected to the body; and
a pair of deformable, elastic components that are an integral part of the articulating arms, wherein an exposed surface of each of the elastic components has one or more geometric surface structures to allow for surface deformations along the exposed surface at the level of an interface between the device and the second object when the device is securely attached to the second object by clamping the articulating arms thereto.

28. The device of claim 27, wherein the geometric surface structures are selected from a group consisting of flexible teeth, flexible upstanding suction cups, and flexible protrusions.

29. The device of claim 27, wherein the articulating arms are constructed so that they can flex.

30. The device of claim 29, wherein the articulating arm includes an adjustable member which is operatively connected to the articulating arm such that rotation of the adjustable member causes flexing and bending of the articulating arm.

31. The device of claim 27, wherein the elastic components and the articulating arms are formed in situ in a common mold.

32. The device of claim 31, wherein the elastic component is formed of a different material or the same material used to form the articulating arm.
